(19) United States
(12) Patent Application Publication
(10) Pub. No.: US 2010/0202985 A1
(43) Pub. Date: Aug. 12, 2010

(54) SUNSCREEN COMPOSITIONS INCLUDING PARTICULATE SUNSCREEN ACTIVES THAT EXHIBIT BOOSTING OF SUN PROTECTION FACTOR

(75) Inventor: Ashoke K. SenGupta, Barrington, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: AMCOL International Corporation, Hoffman Estates, IL (US)

(21) Appl. No.: 12/369,514

(22) Filed: Feb. 11, 2009

(51) Int. Cl. A61K 8/29; A61Q 17/04; A61K 8/18

(52) U.S. Cl. 424/59

(57) ABSTRACT

The present invention relates to emulsion-based sunscreen compositions, including only inorganic ultraviolet radiation (UV)absorbers. Specifically, it relates to sunscreen compositions in the form of oil-in-water (O/W) and water-in-oil (W/O) emulsions that contain inorganic UV-absorbers and an SPF-boosting additive. The sunscreen compositions include an emulsion of an oil phase and a water or hydrophilic liquid phase, an inorganic UV-absorber in the oil phase, and an additive in the water or hydrophilic liquid phase, the additive capable of boosting the sun protection factor (SPF) of the sunscreen composition by at least 20%.

### BACKGROUND OF THE INVENTION

It is well recognized that solar ultraviolet (UV) radiation poses a serious threat to human skin, which may range from the short term hazard like erythema, i.e., sunburn, to long term hazards like skin cancer and/or premature aging of the skin. UV radiation having a wavelength of 290 nm to 320 nm, generally referred to as UVB radiation, is known to cause erythema. In addition, numerous studies point to exposure of unprotected skin to UV radiation having a wavelength of from 320 nm to 400 nm, generally referred to as UVA radiation, as being the primary cause of skin cancer. It is essential, therefore, that skin is protected from both UVA and UVB radiations to avoid the long and short term deleterious effects of solar radiation.

The sun protection factor (SPF) rating system has been developed to help consumers select the appropriate sun protection product for any given outdoor activity involving exposure to the sun. The SPF rating corresponds to a multiplying factor by which the duration of protection by a properly applied sunscreen exceeds the exposure time that causes unprotected skin to darken. Thus, with proper application of an SPF 15 product, a person should be able to remain in the sun without skin darkening for fifteen times the usual unprotected duration.

In recent years, due to the increased public awareness of UV radiation hazards, the use of sun protection products has grown considerably, with consumers preferring products that have high SPF ratings and offer protection over the entire range of UV radiation, i.e., from 290 nm to 400 nm. These products typically contain certain UV-absorbers that are approved for use in sunscreen compositions by regulatory agencies (for example, US Food and Drug Administration (FDA) in the USA and COLIPA in the European Union). These approved UV-absorbers are either organic compounds, called herein as organic UV-absorbers or sunscreens, or inorganic compounds, called herein as inorganic UV-absorbers or sunscreens. At present, approved inorganic UV-absorbers are inorganic oxides such as titanium dioxide (TiO₂) and zinc oxide (ZnO). Among these inorganic oxide sunscreens, ZnO is capable of absorbing substantial amounts of UVA-radiation, and hence is often used as a UVA-sunscreen.

U.S. Pat. No. 6,500,411 discloses an additive that is capable of boosting the sun protection factor (SPF) of sunscreen compositions that include only organic UV-absorbers as functional sunscreens. The constituent materials for the additive include inorganic oxides such as pigmentary-grade TiO₂ and/or ZnO which can potentially serve as UV-absorbers, however, the amounts of these inorganic oxides added to a sunscreen composition through the said additive at its typical use-level, is much too small for making any significant contribution as an UV-absorber to the SPF-rating of the sunscreen composition. In other words, a composition comprising this additive at its typical use-level, but free of any UV-absorber, cannot provide for any considerable sun protection.

Despite their extensive use in sunscreen products, it is generally recognized that organic sunscreens produce an oily skin-feel and can be skin-irritants. Due to these undesirable features of organic sunscreens, the consumer preference for sunscreen products that contain only inorganic sunscreens is on the rise in recent times. Nonetheless, it is generally considerably difficult, if not impossible, to achieve high SPF ratings with inorganic sunscreens alone (i.e., without the organic sunscreens), unless they are used at relatively high levels. Yet another limitation of inorganic sunscreens is that they tend to cause skin-whitening, when used at sufficiently high levels typically required for realizing high SPF ratings. There is a growing need, therefore, for sunscreen products which contain only inorganic sunscreens, yet which can provide adequate sun protection, without causing any gross skin-whitening. The present invention is directed towards meeting this need, and accordingly discloses methods and compositions for achieving high SPF ratings for sunscreen compositions that contain modest levels of inorganic sunscreens.

### SUMMARY OF THE INVENTION

The present invention relates to emulsion-based sunscreen compositions including only inorganic ultraviolet radiation (UV) absorbers known in the art. Specifically, it relates to sunscreen compositions in the form of oil-in-water (O/W) and water-in-oil (W/O) emulsions that contain inorganic UV-absorbers and an SPF-boosting additive.

The sunscreen compositions include an O/W or W/O emulsion that has an oil phase and a water or hydrophilic liquid phase, an inorganic UV-absorber in the oil phase, and an additive in the water or hydrophilic liquid phase, the additive capable of boosting the sun protection factor (SPF) of the sunscreen composition by at least 20%. The sunscreen composition can further include an inorganic UV-absorber in the water phase. The inorganic UV-absorbers are preferably selected from the group consisting of titanium dioxide, zinc oxide, and mixtures thereof.

The inorganic UV-absorber is preferably about 2%-50% by weight of the composition, more preferably about 4%-15% by weight of the composition, and most preferably about 5%-10% by weight of the composition. The additive is preferably about 0.1%-35% by weight of the composition, more preferably about 0.25%-20% by weight of the composition, and most preferably about 0.5%-10% by weight of the composition.

The additive preferably includes a particulate material. The particulate material is preferably selected from the group consisting of titanium dioxide, zinc oxide, alumina, silica, cerium oxide, bentonite, talc, calcium sulfate, barium sulfate, and mixtures thereof.

The additive preferably includes an interfacially-active polymer capable of adsorbing on i) a surface of the particulate material and ii) an air-water interface, wherein the interfacially-active polymer is capable of reducing the surface tension for the air-water interface by at least 15%, and wherein the interfacially-active polymer is water-soluble or water-dispersible. The interfacially-active polymer is preferably selected from the group consisting of lignosulfonate, lignin, alkali metal salts of humic acid, alkali metal salts of tannic acid, and mixtures thereof.

The sunscreen composition can further include an emulsifier in the water phase, the oil phase, or both water and oil phases.

Unexpectedly, it has now been found that the sunscreen compositions, which contain only inorganic UV-absorbers, exhibit a significant boost in the SPF rating due to the additive contained therein, despite being free of any organic UV-absorbers. The said SPF boosting additive is available from AMCOL International Corporation under the trade-name of Polargel® UV.

### DESCRIPTION OF THE INVENTION

The sunscreen compositions as oil-in-water (O/W) or water-in-oil (W/O) emulsions, described herein, include the following essential components:
i) An oil or an emollient, present in the oil-phase
ii) Water or a hydrophilic organic liquid or mixtures thereof, present in the water/hydrophilic liquid-phase
iii) An emulsifier, present either in the oil-phase or in the water-phase, or in both phases
iv) Inorganic UV-absorber, present either entirely in the oil-phase or in both the oil- and the water-phase
v) An additive that is capable of boosting the SPF of the sunscreen compositions, present in the water-phase

### Oil or Emollient

By oil or emollient is meant a hydrophobic liquid or a hydrophobic gel or waxy solid that has a negligible solubility (<0.5% by weight) in water or a hydrophilic organic liquid. Non-limiting examples consist of various ester solvents including triglycerides, hydrocarbons, fatty alcohols having preferably an alkyl chain length of C₂₀ or higher, and silicone fluids and waxes including ethoxylated and/or propoxylated silicone fluids and waxes. The preferred hydrophobic liquids include isopropyl myristate, isopropyl palmitate, C₁₂₋₁₅ alkyl benzoate, caprylic/capric triglyceride, squalane, cyclomethicone, dimethicone, cetyl dimethicone, isohexadecane, polyisobutene, cetearyl isonanonate, ethylhexyl stearate, ethylhexyl palmitate, diisopropyl adipate, PPG-3 myristyl ether, PPG-11 stearyl ether, and vegetable oils such as sunflower oil, sesame seed oil, and jojoba oil. The preferred hydrophobic gel or waxy solids include petrolatum, arachidyl alcohol, castorlatum, cetyl ricinoleate, myristyl myristate, polyethylene, silicone waxes, ceresin wax, ozokerite wax, and natural waxes such as carnauba wax, candelila wax, beeswax, and lanolin wax, and mixtures thereof. The amount of the oil-phase in the sunscreen compositions is preferably in the range of 5%-90%, more preferably in the range of 10%-75%, and most preferably in the range of 10%-60% by weight of the compositions.

### Hydrophilic Organic Liquid

According to one embodiment, the water-phase or the hydrophilic liquid-phase of the sunscreen emulsions may comprise a hydrophilic organic liquid. Non-limiting examples of a hydrophilic organic liquid which may be included in the water/hydrophilic liquid-phase of the sunscreen emulsions, include glycols such as propylene glycol, butylene glycol, hexylene glycol, and polyethylene glycol, glycerin, water-soluble alcohols, ethoxylated and/or propoxylated dimethicone, ethoxylated glyceryl cocoate, sucrose cocoate, ethoxylated caprylic/capric glycerides, and mixtures thereof. The amount of the water phase or hydrophilic liquid-phase is preferably in the range of 1%-90%, more preferably in the range of 5%-75%, and most preferably in the range of 10%-60% by weight of the sunscreen compositions. The amount of the hydrophilic organic liquid can be range of 0%-90%, more preferably in the range of 1%-75%, and most preferably in the range of 5%-60% by weight of the sunscreen compositions.

### Emulsifier

Any low molecular weight surfactant or polymeric emulsifier known in the art as an emulsifier is suitable for the sunscreen compositions. Preferred O/W emulsifiers include cetyl phosphate, sodium cetearyl sulfate, sodium stearate, sodium lauryl sulfate, methyl glucose sequistearate, polyglyceryl-3 methylglucose distearate, glyceryl stearate citrate, glyceryl stearate, glycol stearate, steareth-25, ceteth-20, ceteareth-15, ceteareth-20, ceteareth-25, steareth-10, steareth-20, polysorbate 80, lauryl glycoside, ceterayl glycoside, and mixtures thereof. Preferred W/O emulsifiers include PEG-30 dipolyhydroxystearate, polyglyceryl-2 dipolyhydroxystearate, cetyl PEG/PPG-10/1 dimethicone, bis-PEG/PPG-14/14 dimethicone, polyglyceryl-4 isostearate, methyl glucose isostearate, glyceryl isostearate, sorbitan stearate, steareth-2, PEG-7, hydrogenated castor oil, and mixtures thereof. The amount of the emulsifier is preferably in the range of 0.1%-30%, more preferably in the range of 0.5%-20%, and most preferably in the range of 1%-10% by weight of the sunscreen compositions.

### Inorganic UV-Absorber

Any inorganic UV-absorber approved for use in sunscreen compositions by regulatory agencies is suitable for the sunscreen compositions. Approved inorganic sunscreens include titanium dioxide and zinc oxide, including hydrophobically surface-modified titanium dioxide and zinc oxide.

The inorganic UV-absorber may be included either entirely in the oil-phase or in both the oil- and the water-phase of the sunscreen compositions. According to one embodiment, the inorganic UV-absorber may be first dispersed in a hydrophobic liquid or an emollient, and the resulting dispersion (called herein as pre-dispersion) is subsequently added to the oil-phase of the sunscreen emulsions of the present invention. When the inorganic UV absorber is also in the water phase, the inorganic UV absorber may be first dispersed in water or a hydrophilic liquid, and the resulting dispersion (called herein as pre-dispersion) is subsequently added to the water/hydrophilic liquid-phase of the sunscreen compositions. A dispersing agent can be used to ensure adequate deflocculation of the inorganic oxide particles. Preferred dispersing agents for dispersing the inorganic UV-absorber in the oil-phase of the sunscreen emulsions include PEG-30 dipolyhydroxystearate, polyglyceryl-2 dipolyhydroxystearate, and PEG-6 distearate. Preferred dispersing agents for the water-phase dispersion of the inorganic oxide particles include polysulfonate (e.g., lignosulfonate), polyacrylate, polyphosphate, and polyphenolate (e.g., sodium humate, sodium tannate).

A pre-dispersion of an inorganic oxide UV-absorber preferably includes an inorganic oxide in an amount of 10%-70%, more preferably 20%-60%, and most preferably 30%-50% by weight of the pre-dispersion. The amount of the dispersing agent is preferably in the range of 0.1%-100%, more preferably in the range of 0.25%-50%, and most preferably in the range of 0.5%-30% by weight of the inorganic UV-absorber contained in the pre-dispersion.

The sunscreen compositions can include particulate UV-absorbers, for example, the particulate UV-absorber available under the tradename of Tinosorb® M (Bisoctrizole; methylene bis-benzotriazolyl tetramethylbutylphenol) from Ciba Specialty Chemicals.

### SPF-Boosting Additive

The sunscreen compositions contain in their water/hydrophilic liquid-phase an additive that is capable of boosting the SPF rating by at least 20%. This additive essentially comprises a particulate material that is virtually insoluble (solubility is less than about 1% by weight) in water or in a hydrophilic liquid or in a hydrophobic liquid, and a water-soluble or water-dispersible, interfacially-active polymer that is capable of adsorbing on the surface of the particulate material. By “interfacially-active” is meant a moiety that is capable of adsorbing at air-water or oil-water interface, and thus reducing the interfacial tension (the surface-tension for a given interface), preferably by at least 15%. Preferred interfacially-active polymers include lignosulfonate, lignin, oxylignin, alkali metal salts of humic acid, alkali metal salts of tannic acid, proteins, hydrophobically-modified water-soluble or water-dispersible polymers, and water-soluble or water-dispersible, amphiphilic copolymers. Preferred particulate materials include titanium dioxide, zinc oxide, alumina, cerium oxide, silica, bentonite, talc, aluminum hydroxide, calcium sulfate, and polymeric latex particles, having a mean particle size in the range of 0.05 micrometers-2 micrometers.

According to a preferred embodiment, the SPF-boosting additive is a water-based dispersion of a particulate material, wherein an interfacially-active polymer serves as a dispersing agent for the particulate material. The amount of the particulate material in the foregoing dispersion is preferably about 1%-70%, more preferably about 5%-50%, and most preferably about 10%-40% by weight of the dispersion. The amount of the interfacially-active polymer is about 0.5%-100%, more preferably about 1%-50%, and most preferably about 2.5%-30% by weight of the particulate material contained in the SPF-booster additive.

The amount of the SPF-booster additive is preferably about 0.1%-35%, more preferably about 0.25%-20%, and most preferably about 0.5%-10% by weight of the sunscreen composition.

Without limiting the scope of the present invention to any particular theory, it is postulated herein that a plausible mechanism for the SPF-boost by the foregoing additive is as follows. The adsorption of an interfacially-active polymer on the surface of the particulate material of the SPF-booster additive, and the resulting coating of the particulate material surface by the polymer, in turn enables the particulate material to adsorb at an oil-water interface. Such interfacial adsorption of the particulate material at the oil-water interface of the emulsion droplets of the sunscreen emulsions, enables the particulate material to hinder or delay the coalescence of the emulsion droplets, as the sunscreen emulsions are rubbed onto the skin, an effect that ultimately ensures a more uniform or even (i.e., less patchy) coverage of the skin by the UV-absorbers contained in the oil-phase of the sunscreen emulsions, resulting in a higher SPF-rating.

The sunscreen compositions may further contain ingredients selected from fatty alcohols having 8 to 22 carbon atoms, opacifiers or pearlescers such as ethylene glycol esters of fatty acids (e.g., ethylene glycol distearate), viscosity and rheology-modifiers selected from polymers, coplymers, cross-polymers, smectite clays, silica, silicate minerals, and inorganic oxides, buffering or pH adjusting chemicals, water-proofing agents, including polymeric and/or wax-based water-proofing agents, plant or botanical or fruit extracts, fragrances, dyes and pigments including iron oxides, preservatives, vitamins, antioxidants, oil-phase thickeners including organoclays and polymeric thickeners, dispersing agents, chelating agents, skin-care actives including anti-wrinkle actives such as retinol and retinol derivatives, skin-lightening actives, anti-acne actives, various organic acids, polyphenols, delivery systems for actives including water- and oil-insoluble polymeric microparticles, polymeric encapsulants, and mixtures thereof.

The following examples are illustrative only and are not to be construed as limitations of the present invention. Many variations thereof are possible without departing from the purview and spirit of the compositions and methods described herein.

### EXAMPLES

### Example I

This example shows pre-dispersions of an inorganic oxide UV-absorber (titanium dioxide, TiO₂), as presented in Table I. The specific TiO₂ product used is UV TITAN M161 (supplier: Kemira), comprising ultrafine TiO₂ particles having a surface coated with alumina and stearic acid. The TiO₂-content of the coated particles is about 75.6% by weight of the total particle.

TABLE I

| | Weight % | | | | |
|---|---|---|---|---|---|
| | Pre- | Pre- | Pre- | Pre- | Pre- |
| Ingredient | dispersion 1 | dispersion 2 | dispersion 3 | dispersion 4 | dispersion 5 |
| Ethylhexyl Stearate | 45 | 31.14 | 41.64 | 15.57 | 26.07 |
| Squalane | 0 | 23.36 | 23.36 | 23.36 | 23.36 |
| Diethylhexyl 2,6 | 0 | 0 | 0 | 15.57 | 15.57 |
| Napthalate | | | | | |
| Polyglyceryl-2 | 5 | 10.5 | 0 | 10.5 | 0 |
| Dipolyhydroxystearate | | | | | |
| TiO₂ (UV TITAN | 50 | 35 | 35 | 35 | 35 |
| M161) | | | | | |

### Example II

This example (Table II) discloses oil-in-water (O/W) sunscreen emulsions, wherein the inorganic oxide UV-absorber is TiO₂ (UV TITAN M161). In Table II, the “control” formulations do not contain the additive, Polargel® UV 1416 (Supplier: AMCOL International Corporation). The particulate material contained in the foregoing additive is pigmentary-grade TiO₂ and sodium bentonite, and the interfacially-active, polymeric surface-modifier for the particulate material is lignosulfonate. At a dosage of 4.5% of this additive, the total amount of solid material added to a sunscreen composition is about 1.3% by weight.

TABLE II
(O/W Emulsions)

| | Weight % | | | | | |
|---|---|---|---|---|---|---|
| Ingredients | Control 1 | Formula 1 | Control 2 | Formula 2 | Control 3 | Formula 3 |
| Polargel ® UV 1416 | 0 | 4.5 | 0 | 4.5 | 0 | 4.5 |
| TiO₂ Pre-dispersion | 20¹ | 20¹ | 20¹ | 20¹ | 14.29³ | 14.29² |
| Deionized Water | 35.95 | 31.45 | 35.95 | 31.45 | 44.38 | 40.38 |
| Butylene Glycol | 3 | 3 | 3 | 3 | 3 | 3 |
| Xanthan Gum (2% | 15 | 15 | 15 | 15 | 10 | 10 |
| solution) | | | | | | |
| Acrylates/C10-30 Alkyl | 0 | 0 | 0 | 0 | 0.5 | 0.5 |
| Acrylate Crosspolymer | | | | | | |
| (Carbopol ® Ultrez 21) | | | | | | |
| Lauryl Glycoside | 14 | 14 | 14 | 14 | 10 | 10 |
| Potassium Cetyl | 0 | 0 | 0 | 0 | 3 | 3 |
| Phosphate (Amphisol K) | | | | | | |
| Squalane | 10 | 10 | 0 | 0 | 4.16 | 4.16 |
| Cyclomethicone | 0 | 0 | 10 | 10 | 0 | 0 |
| C₁₂₋₁₅ Alkyl Benzoate | 0 | 0 | 0 | 0 | 2.5 | 2.5 |
| Ethylhexyl Stearate | 0 | 0 | 0 | 0 | 5.56 | 5.56 |
| Minor Ingredients (e.g., | 2.05 | 2.05 | 2.05 | 2.05 | 2.11 | 2.11 |
| preservative, vitamins, | | | | | | |
| antioxidant, pH- | | | | | | |
| adjustment reagent) | | | | | | |
| In-vivo SPF | 20.47 | 30.7 | 21.93 | 30.7 | 26.17 | 35.82 |

¹TiO₂ Pre-dispersion 1 from Table I
²TiO₂ Pre-dispersion 2 from Table I
³TiO₂ Pre-dispersion 3 from Table I

The manufacturing procedures used for making the sunscreen emulsions in Table II are in accordance with standard emulsion-making procedures known in the art, involving, in general, the following steps:
i) Combine all water-soluble or water-dispersible ingredients
ii) Combine all oil-soluble or oil-dispersible ingredients
iii) Add the oil-phase (optionally heated) slowly to the water-phase (optionally heated), while homogenizing the batch under high-shear agitation

The in-vivo SPF ratings for the sunscreen compositions in Table II (and in the rest of the Tables presented herein) were determined based on 3-subject in-vivo SPF testing (carried out at Cantor Laboratory, New York) as per the static SPF testing protocol mandated by the US Food and Drug Administration.

### Example III

This example shows water-in-oil (W/O) sunscreen emulsions, as exemplified in Table III. The two compositions presented in Table III, are similar, except that Control 4 does not contain the SPF-boosting additive, Polargel® UV 1416. In addition, the TiO₂ pre-dispersions and the emulsifiers used in the two formulations differ.

TABLE III
(W/O Emulsion)

| | | Weight % | |
|---|---|---|---|
| Phase | Ingredients | Formula 4 | Control 4 |
| A-Oil | Squalane | 10.16 | 10.16 |
| | Ethylhexyl Stearate | 13.06 | 13.06 |
| | Cyclomethicone | 5 | 5 |
| | Tocopheryl Acetate | 0.1 | 0.1 |
| | Retinyl Palmitate | 0.01 | 0.01 |
| | Polyglyceryl-2 | 5 | 0 |
| | Dipolyhydroxystearate | | |
| | Cetyl PEG/PPG-10/1 Dimethicone | 0 | 5 |
| | TiO₂ Pre-Dispersion | 14.29² | 14.29³ |
| | Microcrystalline Wax | 2 | 2 |
| B-Water | D.I. Water | 41.43 | 45.93 |
| | EDTA | 0.05 | 0.05 |
| | Butylene Glycol | 3 | 3 |
| | Aloe Extract | 0.15 | 0.15 |
| | Ascorbyl Phosphate | 0.03 | 0.03 |
| | Euxyl PE 9010 (Preservative) | 0.97 | 0.97 |
| | Chlorphenesin (Preservative) | 0.25 | 0.25 |
| | Polargel ® UV 1416 | 4.5 | 0 |
| In-vivo SPF | | 36.75 | 26.25 |

²TiO₂ Pre-dispersion 2 from Table I
³TiO₂ Pre-dispersion 3 from Table I

### Example IV

This example shows (Table IV) O/W sunscreen emulsions, which contain a mixture of zinc oxide (ZnO) and TiO₂ as the inorganic oxide UV-absorber, wherein the two inorganic oxides are added to the sunscreen emulsion as separate pre-dispersions (as part of the emulsion's oil-phase). The specific ZnO product used is a ZnO dispersion product (Tradename: TNC 65F25) from Kobo Products, Inc., New Jersey.

TABLE IV
(O/W Emulsion)

| | Weight % | |
|---|---|---|
| Ingredients | Formula 5 | Control 5 |
| Deionized Water | 40.51 | 44.91 |
| Acrylates/C10-30 Alkyl Acrylate Crosspolymer | 0.5 | 0.5 |
| (Carbopol ® Ultrez 21) | | |
| Xanthan Gum (2%) | 10 | 10 |
| Euxyl PA 9010 (Preservative) | 0.97 | 0.97 |
| EDTA | 0.05 | 0.05 |
| Butylene Glycol | 3 | 3 |
| Aloe Extract | 0.15 | 0.15 |
| Ascorbyl Phosphate | 0.03 | 0.03 |
| Chlorphenesin (Preservative) | 0.25 | 0.25 |
| Lauryl Glycoside | 10 | 10 |
| Potassium Cetyl Phosphate (Amphisol K) | 3 | 3 |
| 50% NaOH | 0.42 | 0.42 |
| Squalane | 2 | 2 |
| Ethylhexyl Stearate | 2.68 | 2.68 |
| Diethylhexyl 2,6 Napthalate | 2.78 | 2.78 |
| Tocopheryl Acetate | 0.1 | 0.1 |
| Retinyl Palmitate | 0.01 | 0.01 |
| TiO₂ Dispersion | 14.29⁴ | 14.29⁵ |
| Kobo ZnO Dispersion (65% ZnO) | 4.76 | 4.76 |
| Polargel ® UV 1416 | 4.5 | 0 |
| In-vivo SPF | 40.93 | 31.5 |

⁴TiO₂ Pre-dispersion 4 from Table I
⁵TiO₂ Pre-dispersion 5 from Table I

### Example V

This example shows O/W sunscreen emulsions, corresponding to different methods of incorporating a combination of TiO₂ and ZnO as UV-absorbers in the sunscreen compositions. In Table V, Formula 6 contained a pre-dispersion (in squalane) of a mixture of TiO₂ (UV TITAN M161 from Kemira) and ZnO (XZ-MS4 from Kobo Products), wherein the amounts of the TiO₂ and ZnO products in the pre-dispersion, respectively, are 18% and 30% by weight. In this pre-dispersion, polyglyceryl-2 dipolyhydroxystearate was used as the dispersing agent at an amount of about 14%, based on the total weight of TiO₂ and ZnO particles contained therein. Formula 7 contained the TiO₂ pre-dispersion 2 (in Table I), while the SPF-boosting additive product (Polargel® UV 3025 from AMCOL International), contained in the formulation, includes a water-based dispersion of ZnO along with relatively small amounts of pigmentary-grade TiO₂ and sodium bentonite as particulate components, and lignosulfonate as an interfacially-active polymer. The amount of ZnO in Polargel® UV 3025 is about 30% by weight.

TABLE V

| | | Weight % | |
|---|---|---|---|
| Phase | Ingredients | Formula 6 | Formula 7 |
| A-Water | Deionized Water | 48.64 | 41.7 |
| | Polargel ® UV 3025 | 0 | 16.67 |
| | Xanthan Gum | 0.5 | 0.5 |
| | EDTA | 0.05 | 0.05 |
| | Euxyl PE 9010 | 0.97 | 0.97 |
| | Butylene Glycol | 3 | 3 |
| | Aloe Extract | 0.15 | 0.15 |
| | Ascorbyl Phosphate | 0.03 | 0.03 |
| | Chlorphenesin | 0.25 | 0.25 |
| | Lauryl Glycoside | 12 | 12 |
| | Sodium Cetearyl Sulfate | 3 | 3 |
| B-Oil | C₁₂₋₁₅ Alkyl Benzoate | 2.5 | 2.5 |
| | Ethylhexyl Stearate | 2.5 | 0 |
| | Diethylhexyl 2,6 Napthalate | 5 | 5 |
| | Squalane | 0 | 5.5 |
| | Tocopheryl Acetate | 0.1 | 0.1 |
| | Retinyl Palmitate | 0.01 | 0.01 |
| | TiO₂ Pre-dispersion | 0 | 8.57² |
| | Mixture of TiO₂ and | 16.67 | 0 |
| | ZnO Pre-dispersion | | |
| Post Add | Polargel UV 1416 | 4.5 | 0 |
| | Citric Acid | 0.13 | 0 |
| In-vivo SPF | | 32.2 | 30 |
| In-vivo PPD | | 8.07 | 9.31 |

²TiO₂ Pre-dispersion from Table I

A measure of skin-protection from the UVA-radiation (320 nm-400 nm of wavelength) is determined via an in-vivo testing method known as Persistent Pigment Darkening (PPD). As shown in Table V, the foregoing sunscreen compositions, when tested for PPD at Cantor Laboratory, New York, showed PPD values that signify considerable protection from the UVA-radiation, which is attributed to UVA-absorbance due to ZnO contained therein.

What is claimed is:

1. A sunscreen composition comprising an emulsion comprising an oil phase and a water or hydrophilic liquid phase, an inorganic UV-absorber in the oil phase, and an additive in the water or hydrophilic liquid phase, the additive capable of boosting the sun protection factor (SPF) of the sunscreen composition by at least 20%.

2. The sunscreen composition of claim 1 further comprising an inorganic UV-absorber in the water phase.

3. The sunscreen composition of claim 1, wherein the inorganic UV-absorber is selected from the group consisting of titanium dioxide, zinc oxide, and mixtures thereof.

4. The sunscreen composition of claim 1, wherein the inorganic UV-absorber is about 2% to about 50% by weight of the composition.

5. The sunscreen composition of claim 4, wherein the inorganic UV-absorber is about 4% to about 15% by weight of the composition.

6. The sunscreen composition of claim 4, wherein the inorganic UV-absorber is about 5% to about 10% by weight of the composition.

7. The sunscreen composition of claim 1, wherein the additive comprises a particulate material.

8. The sunscreen composition of claim 7, wherein the additive comprises an interfacially-active polymer capable of adsorbing on i) a surface of the particulate material and ii) an air-water interface, wherein the interfacially-active polymer is capable of reducing surface tension for the air-water interface by at least 15%, and wherein the interfacially-active polymer is water-soluble or water-dispersible.

9. The sunscreen composition of claim 1, wherein the additive is about 0.1% to about 35% by weight of the composition.

10. The sunscreen composition of claim 1, wherein the additive is about 0.25% to about 20% by weight of the composition.

11. The sunscreen composition of claim 1, wherein the additive is about 0.5% to about 10% by weight of the composition.

12. The sunscreen composition of claim 7, wherein the particulate material is selected from the group consisting of titanium dioxide, zinc oxide, alumina, silica, cerium oxide, bentonite, talc, calcium sulfate, barium sulfate, and mixtures thereof.

13. The sunscreen composition of claim 8, wherein the interfacially-active polymer is selected from the group consisting of lignosulfonate, lignin, alkali metal salts of humic acid, alkali metal salts of tannic acid, and mixtures thereof.

14. The sunscreen composition of claim 1 further comprising an emulsifier in the water phase, the oil phase, or both water and oil phases.
